(19) United States
(12) Patent Application Publication
(10) Pub. No.: US 2016/0374854 A1
(43) Pub. Date: Dec. 29, 2016

(54) SURGICAL APPARATUS AND METHOD OF MAKING THE SAME

(71) Applicant: ART, Ltd., Grand Cayman (KY)

(72) Inventors: Ravi Nallakrishnan, Westmont, IL (US); Takayuki Akahoshi, Tokyo (JP)

(73) Assignee: ART, Ltd., Grand Cayman (KY)

(21) Appl. No.: 15/190,366

(22) Filed: Jun. 23, 2016

Related U.S. Application Data
(60) Provisional application No. 62/185,051, filed on Jun. 26, 2015.
(60) Provisional application No. 62/187,297, filed on Jul. 1, 2015.

(51) Int. Cl. A61F 9/007; A61M 1/00

(52) U.S. Cl. CPC A61F 9/00736; A61M 1/008; A61M 1/0058; A61M 2207/00

(57) ABSTRACT

An irrigation and aspiration apparatus suited for irrigation and aspiration during an eye surgery procedure includes a hollow aspiration cannula having proximal and distal ends. An outer, aspiration tip is positioned on at least the distal end of the aspiration cannula, and includes a generally spherical body portion defining an aspiration port in fluid communication with an interior passage defined by the aspiration cannula. The aspiration cannula defines an annular retention step at an outer surface thereof spaced from the distal end, with the aspiration tip defining an internal, annular abutment surface for engagement with the annular retention step to maintain the aspiration tip on the cannula in substantially fluid-tight relationship therewith.

### PRIORITY

This application claims priority for U.S. Provisional Patent Applications No. 62/185,051, filed Jun. 26, 2015, and No. 62/187,297, filed Jul. 1, 2015, the contents of both of which are hereby incorporated by reference.

### TECHNICAL FIELD

The present invention relates generally to instruments used in eye surgery and, more particularly to an irrigation/aspiration (I/A) assembly or apparatus used to remove particles from the capsular bag after removal of a lens, with the apparatus including an integrated aspiration cannula and outer aspiration tip.

### BACKGROUND OF THE INVENTION

When the human eye becomes aged, diseased, or injured it may be necessary to remove the natural lens of the eye. Such removal is common for cataract surgery, in which a lens that has become clouded is removed. The removal of the natural lens of the eye will result in the loss or alteration of focused vision of a patient. Therefore, an artificial lens will be necessary to restore the vision of the patient. Some eyes have an oblong, irregularly shaped cornea that causes astigmatism, or blurred vision due to a refractive error in the eye.

The lens of the eye is held within a capsular bag positioned behind the iris in the anterior chamber of the eye. When the lens becomes damaged or diseased, a common surgical technique is to remove the lens and replace it with an artificial intraocular lens (IOL). Removal of the lens is commonly carried out by physical dismemberment of the lens and phacoemulsification, that is, using a needle to which vibrational energy is transmitted by a phacoemulsification hand piece.

During phacoemulsification, the lens is broken into fragments and the fragments are emulsified and then removed from the capsular bag by aspirating the fragments through a cannula formed as part of the phacoemulsification needle. After the lens has been removed, the capsular bag must be cleaned in order to prepare it for the insertion of an IOL. In particular, epithelial and cortical tissue fragments must be removed from the floor and perimeter of the capsular bag. Typically, an irrigation aspiration (I/A) tip is coupled to a hand piece, which may be the phacoemulsification hand piece, for removal of the fragments. The hand piece will typically have an irrigation and aspiration mode specifically for this task.

Complete removal of the cortex is important for several reasons. If the cortex is not completely removed it may cause post-operative inflammation and an increase in intraocular pressure. Incomplete removal of the cortex may also cause decentration or tilting of the IOL which, in turn, would cause a postoperative refractive error or induced astigmatism. This is especially important if the IOL is a multi-focal or toric type. Incomplete removal of the cortex may also result in the formation of after (secondary) cataract which would impair vision and require laser capsulotomy.

The present invention is directed to an irrigation aspiration (I/A) apparatus or assembly which has been specifically configured to promote safe and efficient irrigation and aspiration of the capsular bag attendant to eye surgery such as implantation of an intraocular lens.

### SUMMARY OF THE INVENTION

In accordance with present invention, an improved irrigation aspiration (I/A) apparatus or assembly includes an aspiration cannula, and an outer aspiration tip secured to the aspiration cannula to provide an integrated assembly for performing irrigation and aspiration attendant to eye surgery. Notably, the aspiration tip has been configured to include a ball-shaped, generally spherical distal body portion, which may comprise relatively soft or cushiony material for compatibility with the capsular tissue.

The ball-like, bulbous or rounded shape of the tip of the body portion presents a softer surface with no angles to lessen the possibility of injury to the capsule. The tip can be used to polish the anterior capsule to remove fine cortical residue as well as any remaining viscoelastic material present after implanting the lens. To aspirate the viscoelastic material, the tip is introduced beneath the IOL accomplishing aspiration without stressing the capsular bag or the ciliary zonules or the lens itself. Use of an outer sleeve or cover made from thermoplastic material over a metallic hollow cannula results in an I/A instrument that presents a smooth tip surface for polishing the cortex and also allows the tip to have a port formed therethrough which communicates with the hollow passageway in the metallic cannula. The tip port can be placed at any desired location on the tip and the tip passageway is then extended to meet the cannula passageway at an angle.

In accordance with one aspect of the present invention, the irrigation aspiration (I/A) apparatus comprises a cannula in the form of metallic, hollow aspiration cannula having a proximal end and a distal end, with the cannula defining an interior passage extending from the proximal end to the distal end thereof.

The apparatus further comprises an outer, aspiration tip positioned on at least the distal end of the aspiration cannula. The aspiration tip includes a generally spherical body portion defining an aspiration port in fluid communication with the interior passage defined by the aspiration cannula, and a mounting sleeve portion extending from the body portion. The mounting portion surrounds the distal end of the aspiration cannula in substantially fluid-tight relationship therewith. The aspiration port defined by the body portion of the aspiration tip may be chamfered.

The aspiration cannula defines an annular retention step at an outer surface spaced from the distal end thereof. In turn, the sleeve portion of the outer aspiration tip defines an internal, annular abutment surface in engagement with the annular retention step for maintaining the aspiration tip in position on the distal end of the aspirating cannula.

In one illustrated embodiment, the aspiration port defined by the body portion of the aspiration tip is coaxially aligned with the interior passage defined by the aspiration cannula. In alternative embodiments, the aspiration port defined by the body portion of the aspiration tip is disposed at an acute angle to the longitudinal axis of the interior passage defined by the aspiration cannula. In one embodiment, the aspiration port defined by the body portion of the aspiration tip is disposed at an acute angle of about 45 degrees to the axis of the interior passage defined by the aspiration cannula.

In accordance with another aspect of the present invention, the aspiration port defined by the body portion of the aspiration tip is disposed at an obtuse angle to the axis of the interior passage defined by said aspiration cannula.

In another aspect of the present I/A apparatus, the aspiration cannula defines an annular retention groove extending proximally of the annular retention step, with the mounting sleeve portion of the aspiration tip defining an annular retention collar disposed within the annular retention groove defined by said aspiration cannula.

In another aspect of the I/A apparatus, the mounting portion of the aspiration tip extends proximally of the annular retention step of the aspiration cannula, and comprises a retention sheath fitted to the exterior of the aspiration cannula. In accordance with illustrated embodiments, the retention sheath preferably has a length that is at least five times the effective diameter of the generally spherical body portion of the aspiration tip, and more preferably about six times the effective diameter of the generally spherical body portion of the aspiration tip.

It is contemplated that the body portion of said aspiration tip may be configured to define a roughened surface. The outer aspiration tip of the assembly may comprise relatively soft material, compared to the metallic material from which the cannula is formed, with the aspiration tip being integrated and joined to the cannula such as by overmolding, swaging, crimping, or otherwise shaping the aspiration tip to the aspiration cannula so that the annular abutment surface of the aspiration tip engages the annular retention step defined by the aspiration cannula.

It has been determined that the aspiration cannula configured in accordance with the present invention can have straight or curved configurations to allow for selectable maneuverability in use.

In another aspect of the present invention, a method of making an irrigation and aspiration apparatus is disclosed. The method has the step of providing a hollow aspiration cannula having a proximal end and a distal end, the aspiration cannula defining an interior passage extending from its proximal end to its distal end. The method has the step of forming an annular retention step at an outer surface of the aspiration cannula spaced from the distal end thereof. The method has the further step of mounting an aspiration tip on at least the distal end of the aspiration cannula, the aspiration tip including a generally spherical body portion defining an aspiration port in fluid communication with the interior passage defined by the aspiration cannula, and the aspiration tip including a mounting sleeve portion extending from the body portion. The mounting portion surrounds the distal end of the aspiration cannula in substantially fluid-tight relationship therewith, and the sleeve portion of the aspiration tip defining an internal, annular abutment surface in engagement with the annular retention step for maintaining the aspiration tip in position on the distal end of the aspiration cannula.

Other features and advantages of the present invention will become readily apparently from the following detailed description, the accompanying drawings, and the appended claims.

While the following describes presently preferred embodiments of the present invention, it is to be understood that this description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed.

### BRIEF DESCRIPTION OF THE DRAWINGS

Reference will hereinafter be made to the accompanying drawings which illustrate aspects of the invention, where like reference numerals used in the various figures denote like structural elements, and wherein:

FIG. 1 is a perspective view of a first I/A apparatus embodying the principles of the present invention;

FIG. 2 is a cross-sectional view of the I/A apparatus shown in FIG. 1;

FIG. 3 is a relatively enlarged, partial cutaway perspective view of an outer aspiration tip of the I/A apparatus shown in FIGS. 1 and 2;

FIG. 4 is a cross-sectional view of the aspiration tip shown in FIG. 3;

FIG. 5 is a view similar to FIG. 3 showing chamfering of an aspiration port of the aspiration tip of the first illustrated embodiment according to the present invention;

FIG. 6 is a cross-sectional view of the aspiration tip shown in FIG. 5;

FIG. 7 is an elevation view of another embodiment of the present invention;

FIG. 8 is a cross-sectional view of the embodiment shown in FIG. 7;

FIG. 9 is an elevational view showing surface treatment of a body portion of the aspiration tip shown in FIGS. 7 and 8;

FIGS. 10-13 are cross-sectional views like FIG. 8 showing disposition of the aspiration port of the aspiration tip at various angles for different embodiments of an aspiration tip according to the present invention;

FIG. 14 is an elevational view of another aspiration tip of the I/A apparatus of the present invention;

FIG. 15 is an exploded perspective view of another I/A apparatus of the present invention, wherein the I/A apparatus is shown together with a portion of a vibratory hand piece and an irrigation sleeve; and

FIG. 16 is a cross-sectional view of another I/A apparatus of the present invention.

### DETAILED DESCRIPTION OF THE DRAWINGS

While the present invention is susceptible of embodiment in various forms, there are shown in the drawings and hereinafter described presently preferred embodiments of the invention, with the understanding that the present disclosure is to be considered an exemplification of the invention, and is not intended to limit the invention to the specific embodiments illustrated.

In accordance with present invention, an improved I/A apparatus or assembly includes an aspiration cannula, and an outer aspiration tip secured to the aspiration cannula to provide an integrated assembly for performing irrigation and aspiration attendant to eye surgery. Notably, the aspiration tip has been configured to include a ball-shaped, generally spherical distal body portion, which may comprise relatively soft or cushiony material for compatibility with the capsular tissue.

Use of an outer aspiration tip in the form of a sleeve or cover made from thermoplastic material, over a metallic hollow cannula, results in an I/A apparatus that presents a smooth tip surface for polishing the cortex and also allows the body portion to have a aspiration port formed therethrough which communicates with the hollow passageway in the metallic cannula. The aspiration port can be placed at any desired location on the tip and the tip passageway is then extended to meet the cannula passageway, either in alignment with the cannula passageway, or at an angle thereto.

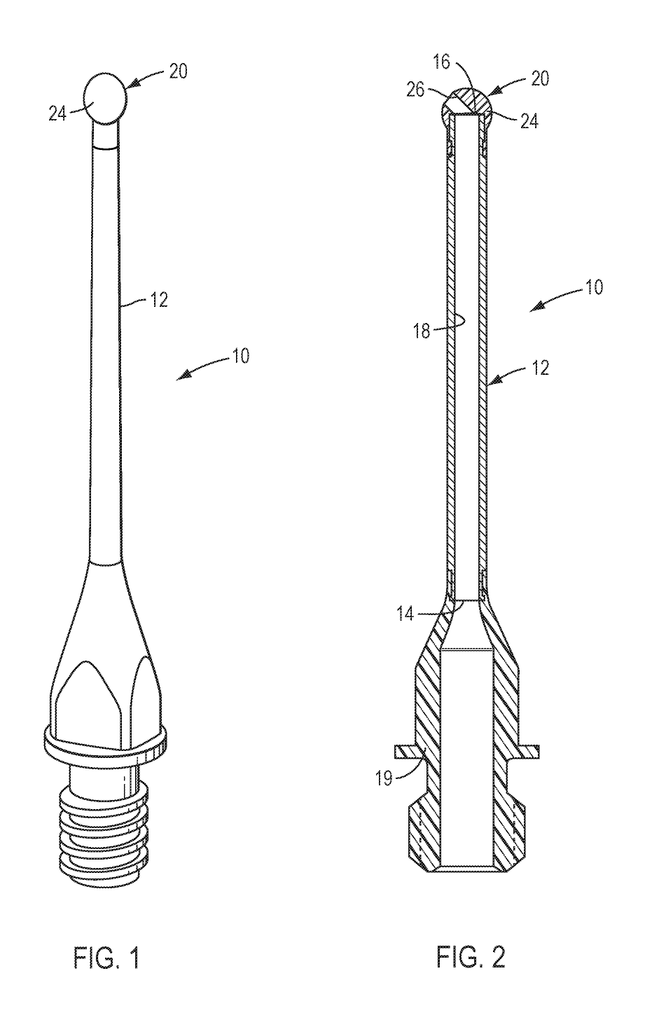

In accordance with the present invention, numeral 10 identifies a miniaturized surgical instrument as described in U.S. Pat. No. 6,491,670, hereby incorporated by reference. As illustrated in FIGS. 1 and 2, an I/A apparatus 10 embodying the principles of the present invention comprises a hollow aspiration cannula 12 having a proximal end 14 and a distal end 16, with the cannula 12 defining an interior passage 18 extending from the proximal end 14 to the distal end 16 thereof. In one currently preferred form, the aspiration cannula 12 is formed from a rigid metal, however, it will be appreciated that the aspiration cannula 12 may be formed from a sufficiently rigid polymer. The I/A apparatus 10 may include a base or hub 19 which is secured to the aspiration cannula 12, typically by over-molding, to thereby operatively connect the I/A apparatus 10 to an associated ultrasonic power source or hand piece, and to an associated vacuum device for creating the desired aspiration through the interior passage of the cannula 12 and through the associated aspiration tip. The hub 19, while preferably formed from a sufficiently rigid polymer, can also be formed integrally with the metallic cannula 12. Features of the cannula 12 can be formed by electro-discharge machining (EDM), laser-etching, press-crimping, injection molding, or by other suitable processes.

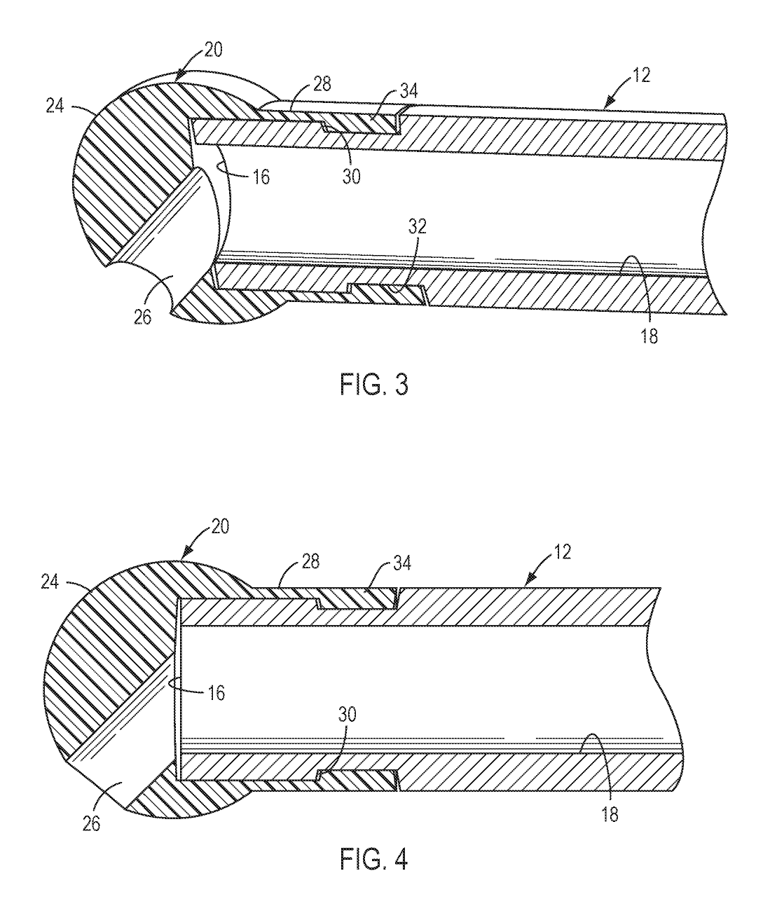

The I/A apparatus further comprises an outer, aspiration tip 20 positioned on at least the distal end of the aspiration cannula 12. The aspiration tip includes a generally spherical body portion 24 defining an aspiration port 26 in fluid communication with the interior passage 18 defined by the aspiration cannula, and a mounting sleeve portion 28 (FIG. 3) extending from the body portion 24. The mounting portion 28 surrounds the distal end 16 of the aspiration cannula 12 in substantially fluid-tight relationship therewith. The aspiration port 26 may typically be provided with a diameter in the range of 0.3 to 0.9 mm.

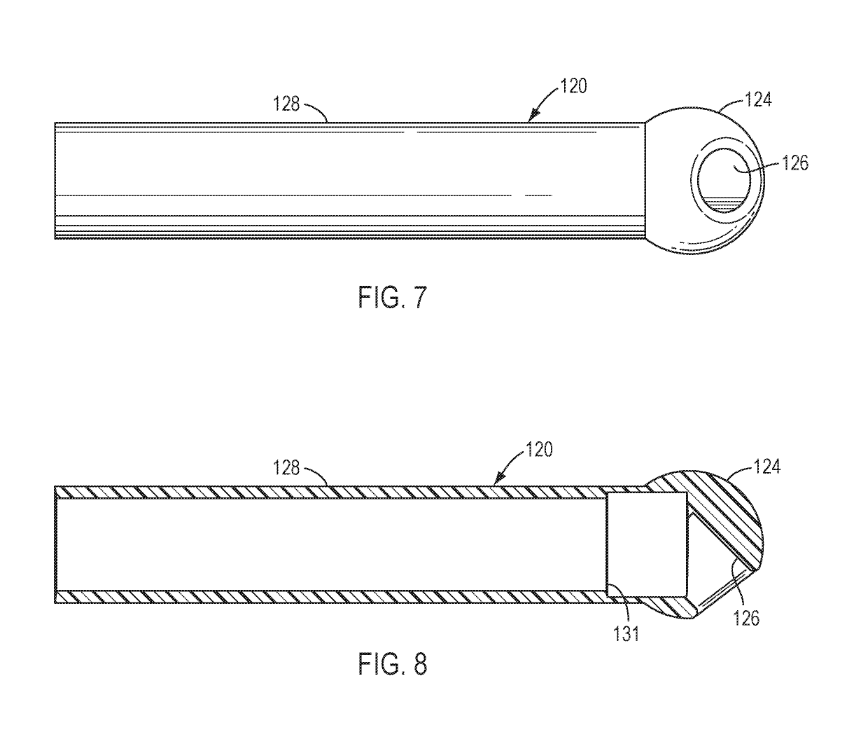

The present I/A apparatus 10 can be configured to promote efficient manufacture, and highly effective and convenient use. In certain embodiments, the aspiration tip 20 of the assembly is configured for overmolding for secure fitment to the associated cannula 12, but can be otherwise affixed such as by crimping, squeezing, swaging, or other manipulation. In certain embodiments, the aspiration tip 20 is provided with an extended mounting sleeve portion, such that the tip 20 can be fitted to the associated cannula 12 in a condom-like fashion, with the mounting sleeve extending proximally from the distal body portion of the aspiration tip 20 to cover a substantial portion of the cannula 12 (e.g., as shown in the various embodiments illustrated in FIGS. 7-14).

The specific configuration of the body portion of the aspiration tip can be varied while keeping with the principles disclosed herein. The aspiration port defined by the body portion can be aligned with (i.e., coaxial) the axis of the interior passage defined by the hollow cannula of the assembly, but need not be configured in this manner. Similarly, the aspiration port can disposed at an acute or obtuse angle to the axis of the interior passage. The material from which the outer aspiration tip is formed can be selected to exhibit a desired degree of softness or tackiness, such a by formation from any of a variety of suitable polymers including silicone-based polymers. A material exhibiting a Shore hardness of 76-82 is preferably employed. It is within the purview of the invention that the body portion of the aspiration tip be subjected to selected surface-finishing, such as roughening by media-blasting or other processes, to create a desired degree of tackiness for the body portion.

In accordance with the first illustrated embodiment of the present invention, as further illustrated in FIG. 3, the aspiration cannula 12 defines an annular retention step 30, and an annular retention groove 32 extending proximally of the annular retention step 30 on an outer surface of the cannula 12, spaced from the distal end 16 of the cannula 12. In turn, the sleeve portion 28 of the outer aspiration tip 20 includes an annular collar 34 which defines an internal, annular abutment surface in engagement with the annular retention step 30 of the cannula 12 for maintaining the aspiration tip 20 in position on the distal end 16 of the aspirating cannula 12. The aspiration tip 20 is preferably formed from a silicone elastomer having sufficient structural strength to retain the shape of tip 20 during surgical procedures.

In this first illustrated embodiment of the I/A apparatus 10, the aspiration port 26 defined by the body portion 24 of the aspiration tip 20 is disposed at an acute angle of about 45 degrees relative to the axis or central axis of the interior passage 18 defined by the aspiration cannula 12. The angle at aspiration port 26 meets the axis of the interior passage 18 defined by cannula 12 is selected to provide the I/A apparatus 10 with a desired and desirable degree of maneuverability in order to allow the tip 20 to be used to aspirate particles from the eye without removing and reorienting the I/A apparatus 10.

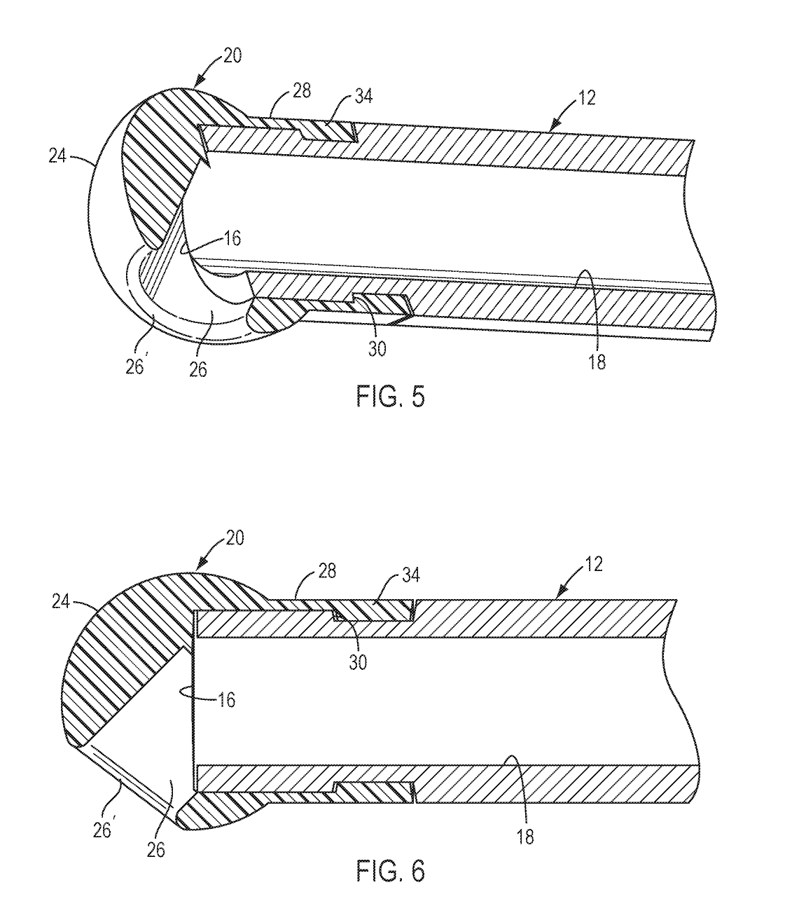

FIGS. 5 and 6 illustrate a variation of the first illustrated embodiment of the present I/A apparatus, wherein the aspiration port 26 of the aspiration tip 20 is chamfered or otherwise shaped at 26′ to present a smooth surface, while avoiding any sharp or pronounced edge portions.

With reference now to FIGS. 7-14, therein are illustrated further embodiments of the present invention. As in the previous embodiments, of the present I/A apparatus, the aspiration cannula defines an annular retention groove extending proximally of the annular retention surface, with the mounting sleeve portion of the associated aspiration tip defining an internal, annular abutment surface in engagement with the annular retention step of the cannula for maintaining the aspiration tip in position on the distal end of the aspirating cannula. In distinction, in these further embodiments, the mounting portion of the aspiration tip extends proximally of the annular retention surface of the aspiration cannula, and comprises a retention sheath fitted over a substantial portion of the exterior of the aspiration cannula. In accordance with illustrated embodiments, the retention sheath preferably has a length that is preferably greater than five times the effective diameter of the generally spherical body portion of the aspiration tip, and more preferably about six times the effective diameter of the generally spherical body portion of the aspiration tip.

With specific reference to FIGS. 7 and 8, as an alternative embodiment of the aspiration tip, designated 120, is illustrated apart from the associated aspiration cannula. The aspiration tip 120. The aspiration tip 120 includes a generally spherical body portion 124 defining an aspiration port 126 configured for fluid communication with the interior passage defined by the associated aspiration cannula. The aspiration tip 120 further includes a mounting sleeve portion 128 extending from the 124 body portion, with the sleeve portion 128 defining an internal, annular abutment surface 131 configured for engagement with a mating annular retention step of the associated aspiration cannula. The mounting portion 128 comprises a retention sheath fitted to the exterior of an aspiration cannula (which would have a flaring shape to retain the mounting portion 128), such that that aspiration tip 120 surrounds the distal end 16 of the aspiration cannula in substantially fluid-tight relationship therewith. As noted, it is presently contemplated that the retention sheath provided by mounting sleeve portion 128 have a length that is about six times the effective diameter of the generally spherical body portion 124, but the precise length of the retention sheath can be varied depending on the application and the polymer used for the tip 120, while keeping with the principles disclosed herein.

As in the previous embodiments, in the embodiment of the aspiration tip 120 shown in FIGS. 7 and 8, the aspiration port 126 defined by the body portion is disposed at an acute angle relative to the axis of the interior passage of the associated aspiration cannula when the aspiration tip 120 is fitted thereto, with the illustrated embodiment showing the aspiration port 126 disposed at an angle of about 45 degrees.

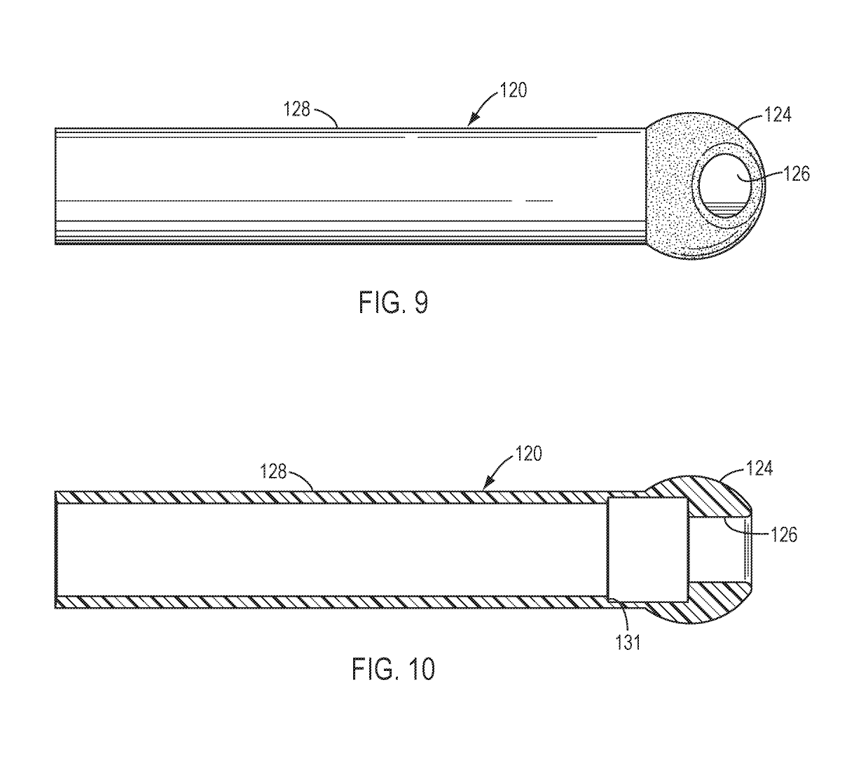

As noted, it is contemplated that the body portion 124 of aspiration tip may be configured to define a roughened surface, or otherwise be selected or configured to present suitable tacky surface. This is illustrated in FIG. 9, which shows an embodiment of the aspiration tip 120 like the shown in FIGS. 7 and 8, but wherein the body portion 124 of the aspiration tip 120 as been roughened, such as by sandblasting or other secondary surface treatment, to provide the desired texture, as indicated at the shading in FIG. 9.

The specific configuration and disposition of the aspiration port of the aspiration tip of the present I/A apparatus can be varied while keeping with the principles disclosed herein. In the embodiment illustrated in FIG. 10, the aspiration port defined by the body portion of the aspiration tip is configured to be coaxially aligned with the axis of the interior passage defined by the associated aspiration cannula.

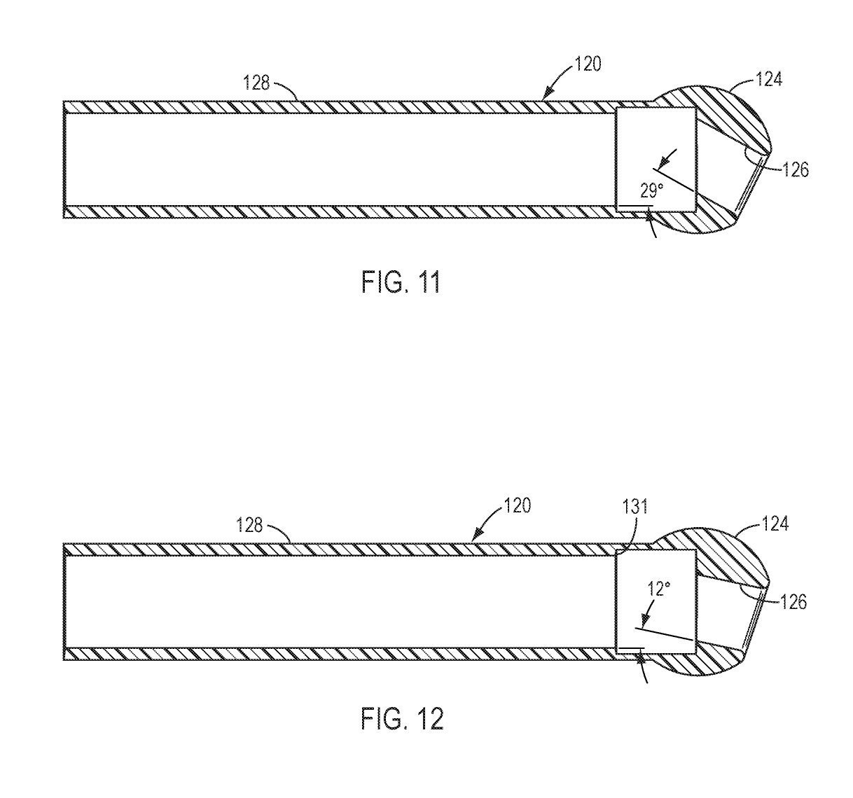

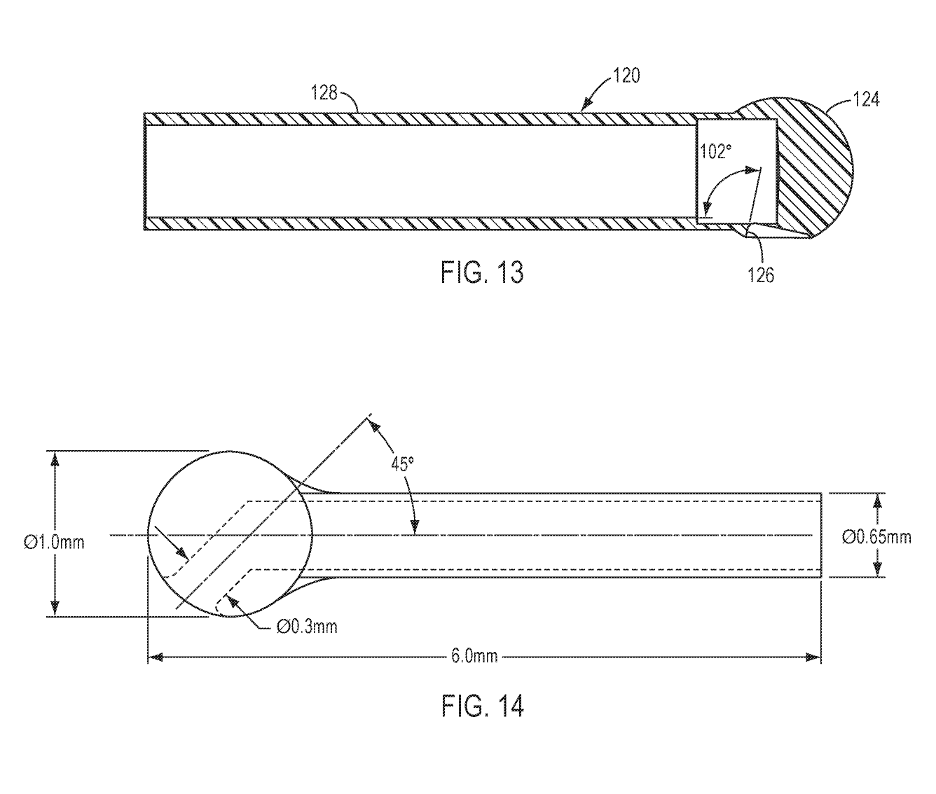

In alternative embodiments, the aspiration port 126 defined by the body portion 124 of the aspiration tip can be configured such that it is disposed at an acute angle to the axis of the interior passage defined by the aspiration cannula. In the embodiment illustrated in FIG. 11, the aspiration port 126 defined by the body portion of the aspiration tip 120 is disposed at an acute angle of about 29 degrees to the interior passage defined by the aspiration cannula, while in the embodiment illustrated in FIG. 12, the aspiration port 126 is disposed at an acute angle of about 12 degrees relative to the axis of the interior passage of the associated aspiration cannula. In contrast, accordance with another aspect of the present invention, as shown in FIG. 13, the aspiration port 126 defined by the body portion of the aspiration tip 120 can disposed at an obtuse angle relative the axis defined by to the interior passage of the aspiration cannula, such as at an obtuse angle of about 102 degrees, as illustrated in FIG. 13.

FIG. 14 further illustrates yet another embodiment of the present I/A apparatus wherein the outer aspiration tip is provided with a sheath-like configuration for fitment to the associated aspiration cannula, with FIG. 14 showing exemplary dimensioning of the aspiration tip. The aspiration tip of this embodiment is greatly enlarged compared to the proximally-extending sheath portion of the tip.

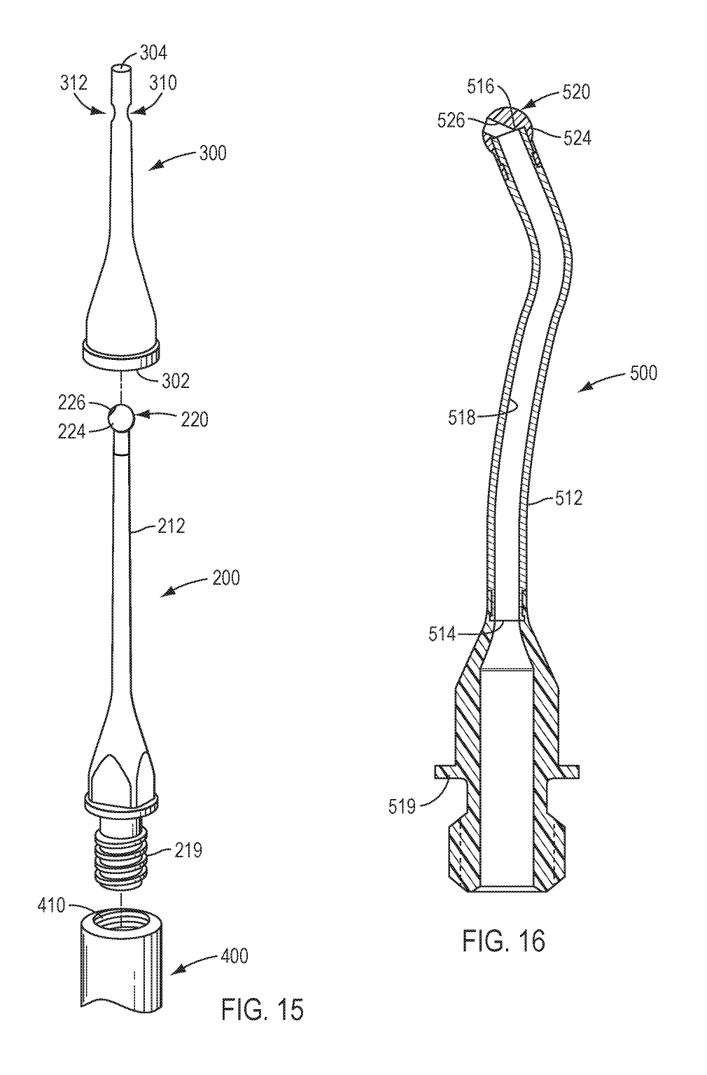

FIG. 15 further illustrates yet another embodiment of the present I/A apparatus 200. The I/A apparatus 200 is similar to the first illustrated embodiment of the I/A apparatus 10, in that the I/A apparatus 200 has a metallic, or rigid polymeric, hollow cannula 212 terminating at one end in a hub 219 and at the other end in a polymeric distal aspiration tip 220. The tip 220 is formed with a generally spherical body portion 224 having an aspiration port 226 formed therein for communicating with the hollow interior of the cannula 212. FIG. 15 further shows an elastomeric and hollow irrigation sleeve 300 that would normally be fitted over the end of the I/A apparatus 200, proximal of the tip 220. The irrigation sleeve 300 has a proximal end 302 and a distal end 304 and one or more irrigation ports 310,312 for providing irrigation fluid to the eye. FIG. 15 further shows a vibratory hand piece 400 having a threaded portion 410, into which the threaded hub 219 is fixed. It will be understood that all of the aforementioned embodiments of the inventive I/A apparatus would be utilized with an appropriate hand piece and irrigation sleeve.

FIG. 16 further illustrates yet another embodiment of the present I/A apparatus 500. The I/A apparatus 500 is similar to the first illustrated embodiment of the I/A apparatus 10, in that the I/A apparatus 500 has a metallic, or rigid polymeric, and hollow cannula 512 terminating at one end in a hub 519 and at the other end in a polymeric distal aspiration tip 520. The tip 520 is formed with a generally spherical body portion 524 having an aspiration port 526 formed therein for communicating with the hollow interior of the cannula 512. The embodiment of the I/A apparatus 500 that is illustrated in FIG. 15 differs from the first illustrated embodiment 10 in that the I/A apparatus 500 has a hollow cannula 512 formed with a bend. Specifically, the cannula 512 is formed with an S-curve, however it will be understood that the cannula 512 may be formed with greater or fewer bends, or bends of greater or lesser degree with respect to the central axis of the I/A apparatus 500.

The inventor has found that embodiments of the I/A apparatus disclosed herein may be advantageously utilized for very soft or so-called “soupy” lenses.

From the foregoing, it will be observed that numerous modifications and variations can be effected without departing from the true spirit and scope of the present invention. It is to be understood that no limitations with respect to the specific embodiment disclosed herein are intended or should be inferred. The disclosure is intended to cover by the appended claims all such modifications as fall within the scope of the claims.

What is claimed is:

1. An irrigation and aspiration apparatus, comprising:
a hollow aspiration cannula having a proximal end and a distal end, and defining an interior passage extending from said proximal end to said distal end; and
an outer, aspiration tip positioned on at least the distal end of said aspiration cannula, said aspiration tip including a generally spherical body portion defining an aspiration port in fluid communication with the interior passage defined by said aspiration cannula, and a mounting sleeve portion extending from said body portion, said mounting portion surrounding the distal end of said aspiration cannula in substantially fluid-tight relationship therewith,
said aspiration cannula defining an annular retention step at an outer surface spaced from said distal end thereof, and said sleeve portion of said aspiration tip defining an internal, annular abutment surface in engagement with said annular retention step for maintaining said aspiration tip in position on the distal end of said aspiration cannula.

2. The irrigation and aspiration apparatus in accordance with claim 1, wherein
said aspiration port defined by said body portion of said aspiration tip is coaxially aligned with an axis of said interior passage defined by said aspiration cannula.

3. The irrigation and aspiration apparatus in accordance with claim 1, wherein
said aspiration port defined by said body portion of said aspiration tip is disposed at an acute angle to an axis of said interior passage defined by said aspiration cannula.

4. The irrigation and aspiration apparatus in accordance with claim 1, wherein
said aspiration port defined by said body portion of said aspiration tip is disposed at an acute angle of about 45 degrees to an axis of said interior passage defined by said aspiration cannula.

5. The irrigation and aspiration apparatus in accordance with claim 1, wherein
said aspiration port defined by said body portion of said aspiration tip is disposed at an obtuse angle to an axis of said interior passage defined by said aspiration cannula.

6. The irrigation and aspiration apparatus in accordance with claim 1, wherein
said aspiration cannula defines an annular retention groove extending proximally of said annular retention surface,
said mounting sleeve portion of said aspiration tip defining an annular retention collar disposed within said annular retention groove defined by said aspiration cannula.

7. The irrigation and aspiration apparatus in accordance with claim 1, wherein
said mounting portion of said aspiration tip extends proximally of said annular retention step of said aspiration cannula and comprises an elongate retention sheath fitted to the exterior of said aspiration cannula.

8. The irrigation and aspiration apparatus in accordance with claim 7, wherein
said sheath has a length that is at least five times the effective diameter of said generally spherical body portion.

9. The irrigation and aspiration apparatus in accordance with claim 1, wherein
said aspiration port defined by said body portion of said aspiration tip is chamfered.

10. The irrigation and aspiration apparatus in accordance with claim 1, wherein
said aspiration cannula is formed with at least one bend between said proximal and distal ends.

11. An irrigation and aspiration apparatus, comprising:
a hollow aspiration cannula having a proximal end and a distal end, and defining an interior passage extending from said proximal end to said distal end; and
an outer, aspiration tip positioned on at least the distal end of said aspiration cannula, said aspiration tip including a body portion defining an aspiration port in fluid communication with the interior passage defined by said aspiration cannula, and a mounting sleeve portion extending from said body portion, said mounting portion surrounding the distal end of said aspiration cannula in substantially fluid-tight relationship therewith,
said aspiration cannula defining an annular retention step at an outer surface of said distal end thereof, and said sleeve portion of said aspiration tip defining an internal, annular abutment surface in engagement with said annular retention step for maintaining said aspiration tip in position on the distal end of said aspirating cannula.

12. The irrigation and aspiration apparatus in accordance with claim 11, wherein
said cannula is metallic and said aspiration tip comprises silicone.

13. The irrigation and aspiration apparatus in accordance with claim 11, wherein
said body portion of said aspiration tip defines a roughened surface.

14. The irrigation and aspiration apparatus in accordance with claim 11, wherein
said aspiration tip is overmolded on said aspiration cannula so that said annular abutment surface engages said annular retention step.

15. The irrigation and aspiration apparatus in accordance with claim 11, wherein
said aspiration tip body portion is generally spherical.

16. The irrigation and aspiration apparatus in accordance with claim 11, further comprising
a hub formed on said proximal end of said aspiration cannula for mounting said irrigation and aspiration apparatus to a hand piece.

17. The irrigation and aspiration apparatus in accordance with claim 16, wherein said hub is formed from a polymer.

18. The irrigation and aspiration apparatus in accordance with claim 11, wherein said aspiration cannula is formed from a polymer that is substantially more rigid than a polymer from which said aspiration tip is formed.

19. A method of making an irrigation and aspiration apparatus, the method comprising the steps of:
providing a hollow aspiration cannula having a proximal end and a distal end, and defining an interior passage extending from said proximal end to said distal end;
forming an annular retention step at an outer surface of said aspiration cannula spaced from said distal end thereof; and
mounting an aspiration tip on at least the distal end of said aspiration cannula, said aspiration tip including a generally spherical body portion defining an aspiration port in fluid communication with the interior passage defined by said aspiration cannula, and a mounting sleeve portion extending from said body portion, said mounting portion surrounding the distal end of said aspiration cannula in substantially fluid-tight relationship therewith, and said sleeve portion of said aspiration tip defining an internal, annular abutment surface in engagement with said annular retention step for maintaining said aspiration tip in position on the distal end of said aspiration cannula.

20. The method of claim 19, further comprising the step of crimping said mounting sleeve portion of said aspiration tip at said distal end of said aspiration cannula.
